(19) United States
(12) Patent Application Publication
(10) Pub. No.: US 2017/0290887 A1
(43) Pub. Date: Oct. 12, 2017

(54) CELL BASED THERAPY FOR FEMALE SEXUAL DYSFUNCTION

(71) Applicant: CREATIVE MEDICAL TECHNOLOGIES, INC., PHOENIX, AZ (US)

(72) Inventors: Thomas Ichim, San Diego, CA (US); Amit Patel, Salt Lake City, UT (US)

(21) Appl. No.: 15/481,249

(22) Filed: Apr. 6, 2017

Related U.S. Application Data
(60) Provisional application No. 62/319,753, filed on Apr. 7, 2016.

(51) Int. Cl. A61K 38/18

(52) U.S. Cl. CPC A61K 38/1866; A61K 35/28

(57) ABSTRACT

Disclosed are compositions of matter, therapeutic means, and methods of use for the treatment of female sexual dysfunction associated with vasculogenic degeneration. In one embodiment the invention teaches the use of systemic, or localized platelet rich plasma for restoration of vasculogenic responsiveness to neural signals associated with sexual function in females

### CROSS-REFERENCE TO RELATED APPLICATIONS

The present invention claims priority to U.S. Provisional Application No. 62/319,753, filed Apr. 7, 2016, which is hereby incorporated in its entirety including all tables, figures, and claims.

### FIELD OF THE INVENTION

The invention pertains to the field of regenerative medicine, more particularly, the invention relates to the field of stimulation of vasculogenesis in females lacking appropriate biological responses to neural stimuli associated with sexual desire.

### BACKGROUND OF THE INVENTION

Male sexual disorders including impotency has attracted significant attention, including the development of PDE5 inhibitors such as Viagra. In contrast, there is a lack of therapies for treating female sexual dysfunction. Female sexual dysfunction such as disorders of sexual desire, arousal or orgasm is a common problem, affecting up to 43% of all women (Pauls et al, Obstret Gynecol Surv, 60(3):3196-205). Both biological and psychological factors contribute to FSD. Available treatments include psychological counseling to pairs or individuals. Where side effects of medication contributes to FSD, altering medication or dosage may help. However, there is a need for improved treatment of FSD, more importantly in biological cases of FSD there are no available treatments. During sexual arousal of the female, vasocongestion of the pelvic region leads to engorgement of the genitalia with blood leading to swelling of the external genitalia and erection of the clitoris. This is accompanied by lubrication of the vagina. In the female, the corpus cavernosa are two paired symmetrical extensions of the clitoris and engorgement of these is an important step during sexual arousal of the female. In a subset of females, lack of endothelial responsiveness to neural stimuli is causative of inability to respond to neural stimuli and as part of a feedback loop to lack sexual desire. While PDES inhibitors have shown some benefit in females (Gao et al. Int J Gynaecol Obstet. 2015 Dec 17. pii: S0020-7292(15)007), as in males, smooth muscle damage/inefficiency surrounding the vasculature, as well as unresponsiveness to neurological stimuli, result in inefficacy of PDE5 inhibitors.

### DESCRIPTION OF THE INVENTION

The invention teaches the use of platelet rich plasma, as well as mesenchymal stem cell generated regenerative factors, for the stimulation/regeneration of smooth muscle, and vasculature associated with clitoral function in females. In particular, using methods described in U.S. Pat. No. 8,372,797, and incorporated by reference, the invention teaches that local, or systemic administration of regenerative factors generated by cells with regenerative potential, or said cells with regenerative potential themselves are useful in restoring, rejuvenating, or increasing sensitivity to female tissues associated with sexual function, in one embodiment being clitoral tissue.

For the practice of the invention, means of stimulating production of regenerative factors from regenerative cells are known in the art and include exposure to agents resembling danger signals, or molecular signals associated with tissue damage, said signals include toll like receptor agonists, ppar agonists, NOD activators, and inflammatory cytokines. Other signals associated with cellular damage include exposure to clotting factors, mechanical stress, hyposmotic stress, hyperosmotic stress, activators of NF-kappa B and other means associated with alterations of normal physiological balance in the body to which mesenchymal stem cells, or other regenerative cells in the body respond to. In certain aspects, the cells can be selected either alone or in combination from a group that includes: stem cells, committed progenitor cells, and differentiated cells. The stem cells can be selected from a group that includes: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells and the like. In certain aspects, the embryonic stem cells can be totipotent, and can express one or more antigens selected from a group that includes: stage-specific specific embryonic antigens (SSEA) 3, SSEA 4, TRa-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT) and the like.

In certain aspects, the cord blood stem cells can be multipotent and capable of differentiating into endothelial, smooth muscle, and neuronal cells. The cord blood stem cells can be identified based on expression of one or more antigens selected from a group that includes: SSEA-3, SSEA-4, CD9, CD34, c-kit, OCT-4, Nanog, and CXCR-4 and the like. Further, the cord blood stem cells selected may not express one or more markers selected from a group that includes: CD3, CD34, CD45, and CD11b and the like. In certain aspects, the placental stem cells can be isolated from the placental structure, and can be identified based on expression of one or more antigens selected from a group that includes: Oct-4, Rex-1, CD9, CD13, CD29, CD44, CD166, CD90, CD105, SH-3, SH-4, TRA-1-60, TRA-1-81, SSEA-4 and Sox-2 and the like.

In certain aspects, the bone marrow stem cells can be bone marrow mononuclear cells, and can be selected based on the ability to differentiate into one or more of the following cell types: endothelial cells, smooth muscle cells, and neuronal cells. The bone marrow stem cells can be selected based on expression of one or more of the following antigens: CD34, c-kit, flk-1, Stro-1, CD105, CD73, CD31, CD146, vascular endothelial-cadherin, CD133 and CXCR-4. Further, the bone marrow stem cells can be enriched for expression of CD133. In certain aspects, the amniotic fluid stem cells can be isolated by introduction of a fluid extraction means into the amniotic cavity under ultrasound guidance. The amniotic fluid stem cells can be selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1. Further, the amniotic fluid stem cells can be selected based on lack of expression of one or more of the following antigens: CD34, CD45, and HLA Class II. In certain aspects, the neuronal stem cells can be selected based on expression of one or more of the following antigens: RC-2, 3CB2, BLB, Sox-2hh, GLAST, Pax 6, nestin, Muashi-1, NCAM, A2B5 and prominin.

In certain aspects, the circulating peripheral blood stem cells can be characterized by ability to proliferate in vitro for a period of over 3 months. Further, the circulating peripheral blood stem cells can be characterized by expression of CD34, CXCR4, CD117, CD113, and c-met. Further, the circulating peripheral blood stem cells may lack substantial expression of differentiation associated markers, such as, for example CD2, CD3, CD4, CD11, CD11a, Mac-1, CD14, CD16, CD19, CD24, CD33, CD36, CD38, CD45, CD56, CD64, CD68, CD86, CD66b, and HLA-DR and the like.

In certain aspects, the mesenchymal stem cells express one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1. Further, the mesenchymal stem cells may not express substantial levels of HLA-DR, CD117, and CD45. In certain aspects, the mesenchymal stem cells can be derived from a group selected of: bone marrow, adipose tissue, umbilical cord blood, placental tissue, peripheral blood mononuclear cells, differentiated embryonic stem cells, and differentiated progenitor cells.

In certain aspects, the germinal stem cells express markers selected from a group that includes: Oct4, Nanog, Dppa5 Rbm, cyclin A2, Tex18, Stra8, Daz1, beta1- and alpha6-integrins, Vasa, Fragilis, Nobox, c-Kit, Sca-1 and Rex1 and the like.

In certain aspects, the adipose tissue derived stem cells express markers selected from a group that includes: CD13, CD29, CD44, CD63, CD73, CD90, CD166, Aldehyde dehydrogenase (ALDH), and ABCG2 and the like. The adipose tissue derived stem cells can be a population of purified mononuclear cells extracted from adipose tissue capable of proliferating in culture for more than 1 month.

In certain aspects, the exfoliated teeth derived stem cells express markers selected from a group that includes: STRO-1, CD146 (MUC18), alkaline phosphatase, MEPE, and bFGF and the like.

In certain aspects, the hair follicle stem cells express markers selected from a group that includes: cytokeratin 15, Nanog, and Oct-4 and the like, and can be capable of proliferating in culture for a period of at least one month. The hair follicle stem cells may secrete one or more of the following proteins when grown in culture: basic fibroblast growth factor (bFGF), endothelin-1 (ET-1) and stem cell factor (SCF).

In certain aspects, the dermal stem cells express markers selected from a group that includes: CD44, CD13, CD29, CD90, and CD105 and the like, and can be capable of proliferating in culture for a period of at least one month.

In certain aspects, the parthenogenically derived stem cells can be generated by addition of a calcium flux inducing agent to activate an oocyte followed by enrichment of cells expressing markers selected from a group that includes SSEA-4, TRA 1-60 and TRA 1-81 and the like.

In certain aspects, the reprogrammed stem cells can be selected from a group that includes: cells subsequent to a nuclear transfer, cells subsequent to a cytoplasmic transfer, cells treated with a DNA methyltransferase inhibitor, cells treated with a histone deacetylase inhibitor, cells treated with a GSK-3 inhibitor, cells induced to dedifferentiate by alteration of extracellular conditions, and cells treated with various combination of the mentioned treatment conditions. In certain aspects, the nuclear transfer can include introducing nuclear material to a cell substantially enucleated, the nuclear material deriving from a host whose genetic profile is sought to be dedifferentiated. The cytoplasmic transfer can include introducing cytoplasm of a cell with a dedifferentiated phenotype into a cell with a differentiated phenotype, such that the cell with a differentiated phenotype substantially reverts to a dedifferentiated phenotype. The DNA demethylating agent can be selected from a group that includes: 5-azacytidine, psammaplin A, and zebularine and the like. The histone deacetylase inhibitor can be selected from a group that includes: valproic acid, trichostatin-A, trapoxin A and depsipeptide and the like.

In certain aspects, the side population cells can be identified based on expression multidrug resistance transport protein (ABCG2) or ability to efflux intracellular dyes such as rhodamine-123 and or Hoechst 33342. The side population cells can be derived from a tissue selected from the group that includes: pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue and the like. In certain aspects, the committed progenitor cells can be selected from a group that includes: endothelial progenitor cells, neuronal progenitor cells, and hematopoietic progenitor cells and the like. Further, the committed endothelial progenitor cells can be purified from the bone marrow, or from peripheral blood, such as from peripheral blood of a patient whose committed endothelial progenitor cells can be mobilized by administration of a mobilizing agent or therapy. The mobilizing agent can be selected from a group that includes: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA-reductase inhibitors and small molecule antagonists of SDF-1 and the like. Further, the mobilization therapy can be selected from a group that includes: exercise, hyperbaric oxygen, autohemotherapy by ex vivo ozonation of peripheral blood, and induction of SDF-1 secretion in an anatomical can be outside of the bone marrow and the like. In certain aspects, the committed endothelial progenitor cells express markers selected from a group that includes: CD31, CD34, AC133, CD146 and flk1 and the like.

In certain aspects, the committed hematopoietic cells can be purified from the bone marrow, or from peripheral blood, such as from peripheral blood of a patient whose committed hematopoietic progenitor cells can be mobilized by administration of a mobilizing agent or therapy. The mobilizing agent can be selected from a group that includes: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA-reductase inhibitors and small molecule antagonists of SDF-1 and the like. Further, the mobilization therapy can be selected from a group that includes: exercise, hyperbaric oxygen, autohemotherapy by ex vivo ozonation of peripheral blood, and induction of SDF-1 secretion in an anatomical can be outside of the bone marrow.

In certain aspects, the committed hematopoietic progenitor cells can express the marker CD133 or CD34.

1. A method of alleviating female sexual dysfunction associated with vasculogenic causes comprising the steps of: a) providing a population of regenerative cells; b) stimulating said regenerative cells ex vivo to induce production of regenerative factors; c) identifying a female patient suffering from sexual dysfunction associated with vasculogenic causes; and d) administering said regenerative factors to said female patient in an amount sufficient to alleviate said female sexual dysfunction.

2. The method of claim 1, wherein said regenerative cells are selected from a group consisting of; a) platelets; b) neutrophils; c) monocytes; d) neural cells; e) mesenchymal stem cells; f) hematopoietic stem cells; g) pluripotent stem cells; and h) smooth muscle cells.

3. The method of claim 2, wherein said platelets are stimulated to coagulate in a manner capable of releasing regenerative factors.

4. The method of claim 3, wherein said activated platelets are in a solution of platelet rich plasma.

5. The method of claim 2, wherein said neutrophils are stimulated in a manner to release neutrophil extracellular traps.

6. The method of claim 5, wherein said neutrophil extracellular traps are cultured with mesenchymal stem cells to induce production of regenerative factors from said mesenchymal stem cells.

7. The method of claim 6, wherein said regenerative factors are composed of at least 10 ng/ml VEGF.

8. The method of claim 7, wherein said regenerative factors are capable of stimulating vasculogenesis.
